(19) United States
(12) Patent Application Publication
(10) Pub. No.: US 2026/0363689 A1
(43) Pub. Date: Nov. 19, 2026

(54) SHAPE MEMORY POLYMERIC FOAM MEDICAL DEVICES WITH STRUCTURAL MEMBER

(71) Applicant: BOSTON SCIENTIFIC SCIMED, INC., MAPLE GROVE, MN (US)

(72) Inventors: Scott Andrew McCullough, Plymouth, MN (US); Philip Litecky, Forest Lake, MN (US)

(73) Assignee: BOSTON SCIENTIFIC SCIMED, INC., MAPLE GROVE, MN (US)

(21) Appl. No.: 19/677,384

(22) Filed: May 14, 2026

Related U.S. Application Data
(60) Provisional application No. 63/806,142, filed on May 15, 2025.

(51) Int. Cl. A61B 17/00; A61L 31/02; A61L 31/04; A61L 31/14; A61B 18/00

(52) U.S. Cl. CPC A61B 17/0057; A61L 31/022; A61L 31/04; A61L 31/146; A61B 2017/00619; A61B 2017/00623; A61B 2017/00632; A61B 2018/00351; A61L 2400/16

(57) ABSTRACT

A left atrial appendage closure (LAAC) device includes a shape memory polymer foam body that expands from a crimped delivery configuration to an expanded configuration. The device incorporates a structural member extending radially within the foam body to assist expansion. The foam body features multiple wings with the structural member extending through them, either internally or externally. The structural member can extend radially from a central hub to the wing peripheries and may include anchors. The structural member can be made of shape memory metal or polymer materials.

### CROSS REFERENCE TO RELATED APPLICATIONS

This application claims the benefit of priority of U.S. Provisional Application No. 63/806,142 filed May 15, 2025, the entire disclosure of which is hereby incorporated by reference.

### TECHNICAL FIELD

The disclosure relates generally to medical devices and more particularly to medical devices that include a shape memory polymeric foam.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted for occluding a patient’s left atrial appendage (LAA). The LAAC device includes a shape memory polymer foam body that is adapted to expand from a crimped configuration for delivery to an expanded configuration after delivery, and a structural member that extends radially within the shape memory polymer foam body and that is adapted to assist in radial expansion of the shape memory polymer foam body.

Alternatively or additionally, the shape memory polymer foam body may include a shaped memory polymer foam body.

Alternatively or additionally, the shape memory polymer foam body may include a plurality of wings formed within the shape memory polymer foam body.

Alternatively or additionally, the structural member may include a plurality of structural components extending through at least some of the plurality of wings.

Alternatively or additionally, the structural member may extend radially within one or more of the plurality of wings.

Alternatively or additionally, the structural member may extend radially from a central hub.

Alternatively or additionally, the structural member may also extend axially within one or more of the plurality of wings.

Alternatively or additionally, the structural member may extend internally within one or more of the plurality of wings.

Alternatively or additionally, the structural member may extend externally to one or more of the plurality of wings.

Alternatively or additionally, the structural member may include a shape memory material.

Alternatively or additionally, the structural member may include a shape memory metal.

Alternatively or additionally, the structural member may include a shape memory polymer.

Alternatively or additionally, the structural member may extend radially through one or more of the plurality of wings to a periphery thereof.

Alternatively or additionally, the structural member may further include an anchor extending radially from the periphery of the one or more of the plurality of wings.

Another example may be found in a medical device. The medical device includes a shape memory polymer foam body that is adapted to expand from a crimped configuration for delivery to an expanded configuration after delivery and a structural member that extends radially within the shape memory polymer foam body and is adapted to assist in radial expansion of the shape memory polymer foam body.

Alternatively or additionally, the structural member may include a shape memory metal.

Alternatively or additionally, the structural member may include a shape memory polymer.

Alternatively or additionally, the medical device may further include an anchor secured to a free end of the structural member.

Alternatively or additionally, the shape memory polymer foam body may be adapted to occlude a left atrial appendage (LAA).

Another example may be found in a left atrial appendage closure (LAAC) device that is adapted for occluding a patient’s left atrial appendage (LAA). The LAAC device includes a shaped memory polymer foam body that is adapted to expand from a crimped configuration for delivery to an expanded configuration after delivery, the shaped memory polymer foam body including a radially extending wing. A structural member extends radially within the radially extending wing and an anchor extends from the structural member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a schematic side view of an illustrative medical device;

FIG. 3 is a schematic top view of an illustrative medical device shown in an expanded configuration;

FIG. 4 is a schematic top view of the illustrative medical device of FIG. 3 shown in a collapsed configuration;

FIG. 5 is a schematic view of a single wing forming part of the illustrative medical device of FIG. 3;

FIG. 6 is a schematic view of the single wing of FIG. 5 including an illustrative structural member extending radially within the wing but not reaching a periphery of the wing;

FIG. 7 is a schematic view of the single wing of FIG. 5 including an illustrative structural member extending radially within the wing to a periphery of the wing, terminating in an anchor;

FIG. 8 is a schematic view of the single wing of FIG. 5 including an illustrative structural member extending both radially and axially within the wing to a periphery of the wing;

FIG. 9 is a schematic view of the single wing of FIG. 5 including an illustrative structural member extending radially within the wing from a central hub structure;

FIG. 10 is a schematic view of a pair of wings, showing an internally extending structural member component within one wing and an externally extending structural member component within a second wing;

FIG. 11 is a schematic view of a first wing including a first structural member component extending radially therethrough and terminating in an anchor;

FIG. 12 is a schematic view of a second wing including a second structural member component extending radially therethrough and terminating in an anchor;

FIG. 13 is a schematic view of an illustrative medical device including the first wing of FIG. 11 and the second wing of FIG. 12; and

FIG. 14 is a schematic top view of the illustrative medical device of FIG. 13.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified .

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

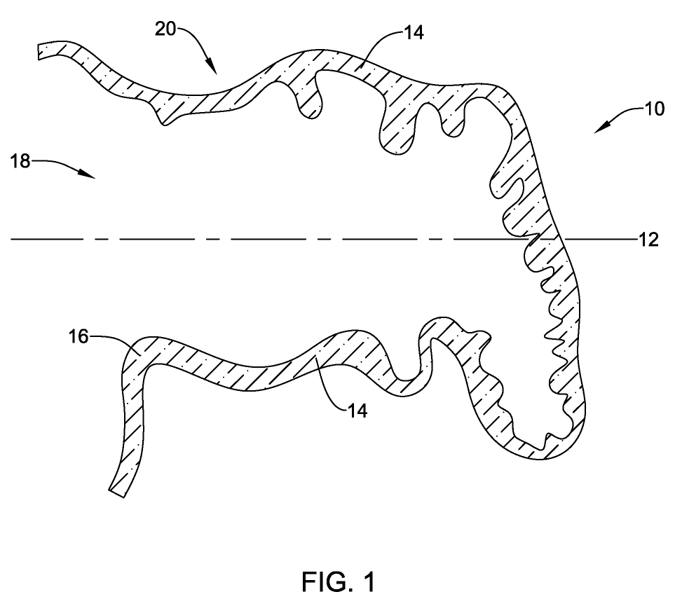

FIG. 1 is a partial cross-sectional view of a left atrial appendage 10. In some cases, a medical device may be adapted to be implanted into the left atrial appendage 10, and to at least partially occlude the left atrial appendage 10. While the medical devices described herein may be used in a variety of different applications, and implanted in a variety of different locations within a patient, these medical devices will be described with respect to being used to at least partially occlude the left atrial appendage 10. In some embodiments, the left atrial appendage (LAA) 10 may have a complex geometry and/or irregular surface area or volume. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

LAAC devices may be made from foam materials such as shape memory polymer foams that have a compressed configuration and an expanded configuration. In some cases, LAAC devices may include a shape memory polymer foam body and structural members that help to further expand the shape memory polymer foam body and thus improve occlusion of the LAA 10. In some cases, the structural members may further include anchors that help to secure the LAAC device in position relative to the LAA 10.

In some instances, a left atrial appendage closure (LAAC) device is adapted for occluding a patient’s left atrial appendage (LAA). The LAAC device includes a shape memory polymer foam body that is adapted to expand from a crimped configuration for delivery to an expanded configuration after delivery, and a structural member that extends radially within the shape memory polymer foam body and that is adapted to assist in radial expansion of the shape memory polymer foam body.

In some cases, the shape memory polymer foam body includes a shaped shape memory polymer foam body. As an example, the shape memory polymer foam body may include a plurality of wings that are formed within the shape memory polymer foam body. In some cases, the structural member may include plurality of structural components that extend through at least some of the plurality of wings. In some cases, the structural member may extend radially within one or more of the plurality of wings. In some cases, the structural member may extend radially from a central hub structure. In some cases, the structural member may also extend axially within one or more of the plurality of wings. The structural member may extend internally within one or more of the plurality of wings. The structural member may extend externally to one or more of the plurality of wings. The structural member may include a shape memory material such as a shape memory metal or a shape memory polymer, for example. In some cases, the structural member may extend radially through one or more of the plurality of wings to a periphery thereof. In some cases, the structural member may further include an anchor that extends radially from the periphery of the one or more of the plurality of wings.

In some instances, a medical device includes a shape memory polymer foam body that is adapted to expand from a crimped configuration for delivery to an expanded configuration after delivery. A structural member extends radially within the shape memory polymer foam body that is adapted to assist in radial expansion of the shape memory polymer foam body.

In some cases, the structural member may include a shape memory metal or a shape memory polymer. In some cases, the medical device may further include an anchor that is secured to a free end of the structural member. As an example, the shape memory polymer foam body may be adapted to occlude a left atrial appendage (LAA).

In some instances, a left atrial appendage closure (LAAC) device is adapted for occluding a patient’s left atrial appendage (LAA). The LAAC device includes a shaped memory polymer foam body that is adapted to expand from a crimped configuration for delivery to an expanded configuration after delivery. In some instances, the shaped shape memory polymer foam body includes at least one radially extending wing. The LAAC device includes a structural member that extends radially within the at least one radially extending wing and an anchor that extends from the structural member.

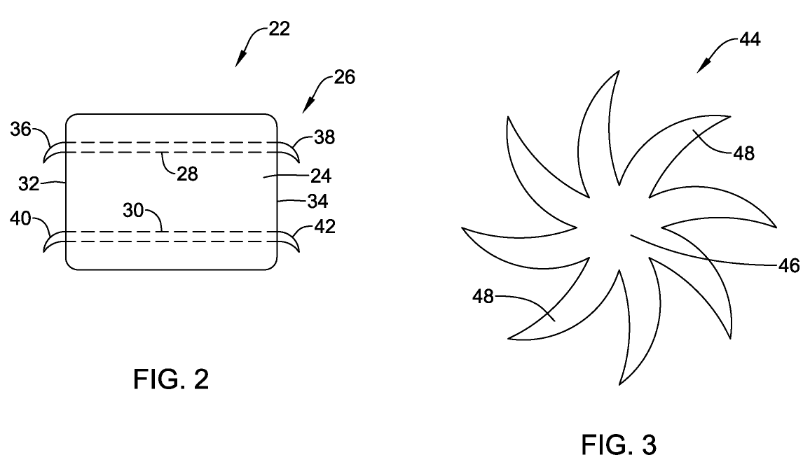

FIG. 2 is a schematic view of an illustrative medical device 22. In some cases, the illustrative medical device 22 may be considered as being an example of a LAAC device that is intended to at least partially occlude part of the left atrial appendage 10. The medical device 22 may have a variety of other uses as part of various medical procedures performed relative to a variety of different body components and systems. The medical device 22 includes a body 24 and a structural member 26 that extends within the body 24. The body 24 may be adapted to expand from a crimped or compressed configuration, such as a configuration in which the medical device 22 may be delivered, and an expanded configuration subsequent to delivery. The expanded configuration may be considered as being a deployed configuration, for example. In some cases, the body 24 is a shape memory polymer foam body. In some cases, the body 24 is a shaped shape memory polymer foam body that has been modified to have a particular three dimensional shape when expanded. The shaped shape memory polymer foam body may be modified in a variety of ways, including but not limited to cutting, such as mechanical cutting and laser cutting, molding, and by bonding together two or more distinct foam components.

In some cases, the structural member 26 may help the body 24 to expand, and in some cases, to expand further than the body 24 would otherwise expand on its own. In some cases, the structural member 26 may provide a radially expansive force that assists in radial expansion of the body 24 and thus the medical device 22. In some cases, the structural member 26 may be formed of a shape memory material such as a shape memory metal or a shape memory polymer. Forming the structural member 26 out of a shape memory material can provide the structural member 26 with a “remembered” configuration. The medical device 22 may be compressed for delivery, for example, which includes compressing both the body 24 and the structural member 26. When compressed, the structural member 26 may be deformed from its “remembered” configuration. When no longer constrained from expanding, the structural member 26 may revert to its “remembered” configuration, thereby radially expanding and providing a radial expansion force to the body 24. In some cases, the structural member 26 may be formed of a non-shape memory material such as spring steel, and may be deformed from its original shape when the medical device 22 is in its compressed configuration, and can spring back to its original shape when no longer constrained from doing so.

The structural member 26 may include multiple components. As shown, the structural member 26 includes a first structural component 28 and a second structural component 30. In some cases, as shown, the first structural component 28 and the second structural component 30 each extend from a first side 32 of the body 24 to a second side 34 of the body 24. In some cases, the first structural component 28 and/or the second structural component 30 may only extend partially across the body 24. As shown, the first structural component 28 terminates at the first side 32 of the body 24 in a first anchor 36 and terminates at the second side 34 of the body 24 in a second anchor 36. The second structural component 30 terminates at the first side 32 of the body 24 in a first anchor 40 and terminates at the second side 34 of the body 24 in a second anchor 42. In some cases, the first structural component 28 and/or the second structural component 30 may not include an anchor, or may include only a single anchor. While the structural member 26 is shown as including the two structural components 28 and 30, in some cases, the structural member 26 may only include one of the structural components 28 or 30. In some cases, the structural member 26 may also include additional structural components. While the structural components 28 and 30 are shown as extending radially within the body 24, in some cases the first structural component 28 and/or the second structural component 30 may include segments that also extend axially within the body 24. In some cases, the first structural component 28 and/or the second structural component 30 may extend at an angle through the body 24 that is neither radial nor axial, but rather at an angle that is intermediate radial and axial. The first structural component 28 and/or the second structural component 30 may extend through the body 24 at any angle. In some cases, the first structural component 28 may extend through the body 24 at a first angle and the second structural component 30 may extend through the body 24 at a second angle that is different from the first angle.

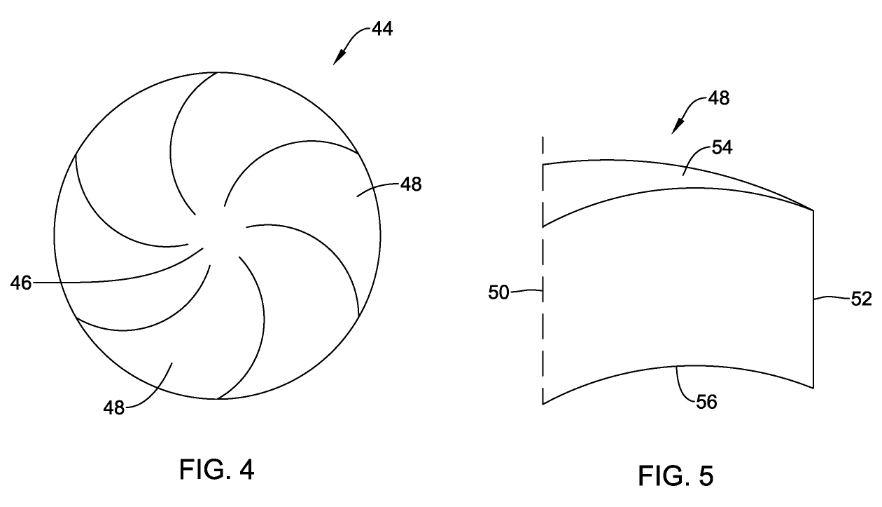

In some cases, the body 24 may be a shaped shape memory polymer foam. FIG. 3 is a schematic top view of an illustrative medical device 44 having a shape memory polymer foam body 46, showing the shape memory polymer foam body 46 in an expanded configuration. FIG. 4 is a schematic top view showing the shape memory polymer foam body 46 in a crimped or compressed configuration. While the medical device 44 may include structural components, the structural components are not shown in this view. The shape memory polymer foam body 46 has been shaped to include a plurality of wings 48. While a total of eight wings 48 are shown, this is merely illustrative. In some cases, the shape memory polymer foam body 46 may have fewer than eight wings 48, such as three wings 48, four wings 48, five wings 48, six wings 48, or seven wings 48. In some cases, the shape memory polymer foam body 46 may have more than eight individual wings 48, such as nine wings 48, ten wings 48, eleven wings 48, twelve wings 48, or even more than twelve wings 48. FIG. 4 shows how the wings 48 are compressed together when in the crimped or compressed configuration.

FIG. 5 is a schematic view of a single wing 48. The wing 48 extends radially outwardly from a central region 50, corresponding to where the wing 48 is attached to a central foam portion that is also attached to each of the other wings 48. The wing 48 has a periphery 52 that is opposite the central region 50. In some cases, the wing 48 includes a first surface 54 and a second surface 56. As an example, when a medical device including the wing 48, or including a plurality of wings 48, is implanted within the LAA 10, the periphery 52 will contact the lateral wall 14 of the LAA 10, one of the first surface 54 and the second surface 56 will be part of a proximal side of the medical device, and the other of the first surface 54 and the second surface 56 will be part of a distal side of the medical device. While FIG. 5 does not show any structural member, it will be appreciated that a structural member, including a plurality of structural components, may take any of a variety of forms within a medical device, and within the wing 48.

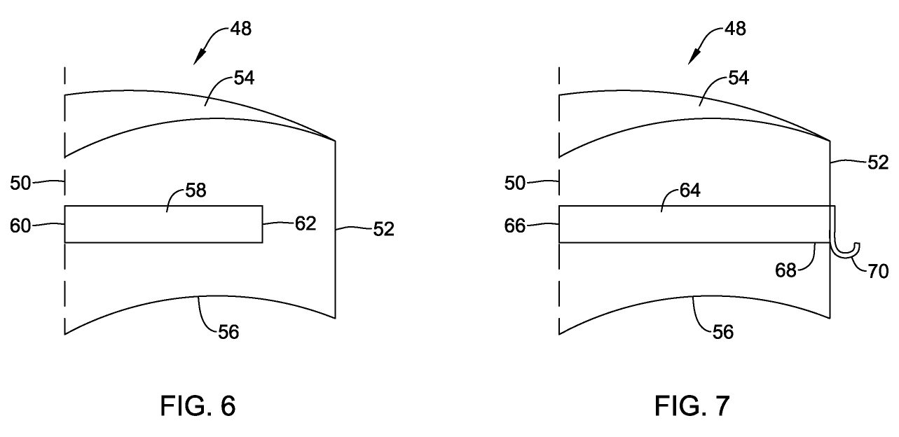

FIG. 6 is a schematic view of the wing 48, showing inclusion of a structural component 58. A medical device including a plurality of the wings 48, such as the medical device 44 shown in FIGS. 3 and 4, may include a structural member that includes one or more structural components such as the structural component 58 in at least some of the plurality of wings 48. In FIG. 6, the structural component 58 extends radially within the wing 48, extending from a first end 60 located at or near the central region 50, to an opposing free end 62. As shown, the free end 62 does not reach the periphery 52 and as a result does not include an anchor.

FIG. 7 is a schematic view of the wing 48, showing inclusion of a structural component 64. A medical device including a plurality of the wings 48, such as the medical device 44 shown in FIGS. 3 and 4, may include a structural member that includes one or more structural components such as the structural component 64 in at least some of the plurality of wings 48. In FIG. 7, the structural component 64 extends radially within the wing 48, extending from a first end 66 located at or near the central region 50, to an opposing free end 68. As shown, the free end 68 reaches the periphery 52 of the wing 48. An anchor 70 is shown extending from the free end 68. The anchor 70 may be formed as part of the structural component 64, or the anchor 70 may be separately formed and then secured to the free end 68 of the structural component 64.

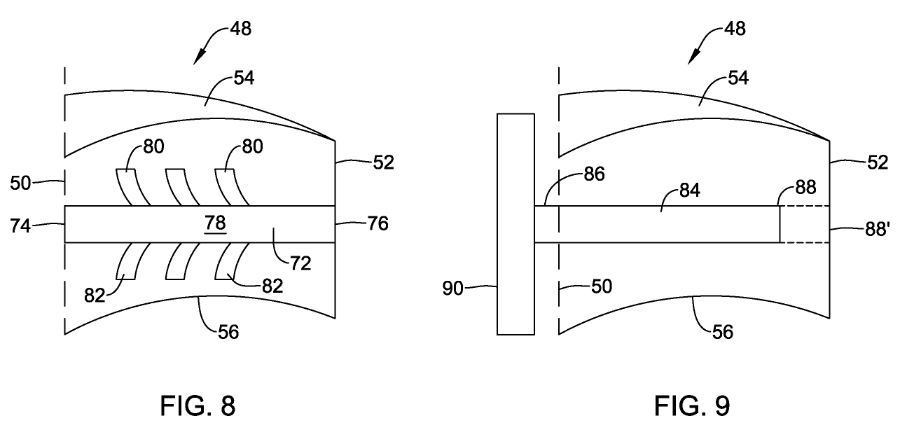

FIG. 8 is a schematic view of the wing 48, showing inclusion of a structural component 72. A medical device including a plurality of the wings 48, such as the medical device 44 shown in FIGS. 3 and 4, may include a structural member that includes one or more structural components such as the structural component 72 in at least some of the plurality of wings 48. In FIG. 8, the structural component 72 includes a portion 78 that extends radially from a first end 74 located at or near the central region 50, to an opposing free end 76. As shown, the free end 76 reaches the periphery 52 of the wing 48. While no anchor is shown, in some cases an anchor may be secured to or be part of the free end 76. In this case, the structural component 72 also includes segments 80 that extend in a first axial direction towards the first surface 54 and segments 82 that extend in a second axial direction towards the second surface 56. The structural component 72 may include any number of segments 80 and any number of segments 82. As an example, the structural component 72 may include zero to ten segments 80 and/or segments 82. The segments 80 and the segments 82 may independently extend at any angle from the portion 78. As an example, the segments 80 and/or the segments 82 may be directed towards the central region, perpendicular to the structural component 72, directed towards the opposing free end, or any combination of these orientations along one or more of the structural components 72. The lengths of each of the segments 80 and the segments 82 may be equal, or may vary.

FIG. 9 is a schematic view of the wing 48, showing inclusion of a structural component 84. A medical device including a plurality of the wings 48, such as the medical device 44 shown in FIGS. 3 and 4, may include a structural member that includes one or more structural components such as the structural component 84 in at least some of the plurality of wings 48. In FIG. 9, the structural component 84 extends radially from a first end 86 located at or near the central region 50, to an opposing free end 88. In this case, the structural component 84 extends radially outwardly from a central hub 90 that extends adjacent to the central region 50. In some cases, the structural component 84 and the central hub 90 are integrally formed. In some cases, the structural component 84 and the central hub 90 are centrally formed and then are secured together via welding, soldering, or adhesive, for example. As shown, the free end 88 does not reach the periphery 52. In some cases, shown in dashed line, the structural component 84 may radially extend through the wing 48 to a free end 88’.

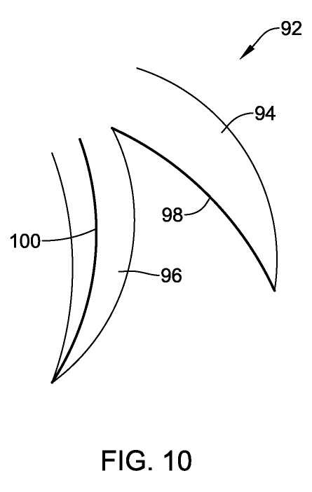

FIG. 10 is a schematic view of a portion of a medical device 92 that includes a first wing 94 and a second wing 96. The first wing 94 and the second wing 96 may each be considered as being examples of the wing 48 described herein. As shown, the first wing 94 includes a structural component 98 that extends along an exterior of the first wing 94. As shown, the second wing 96 includes a structural component 100 that extends within an interior of the second wing 96. The medical device 92 may include combinations of externally located structural components 98 and internally located structural components 100.

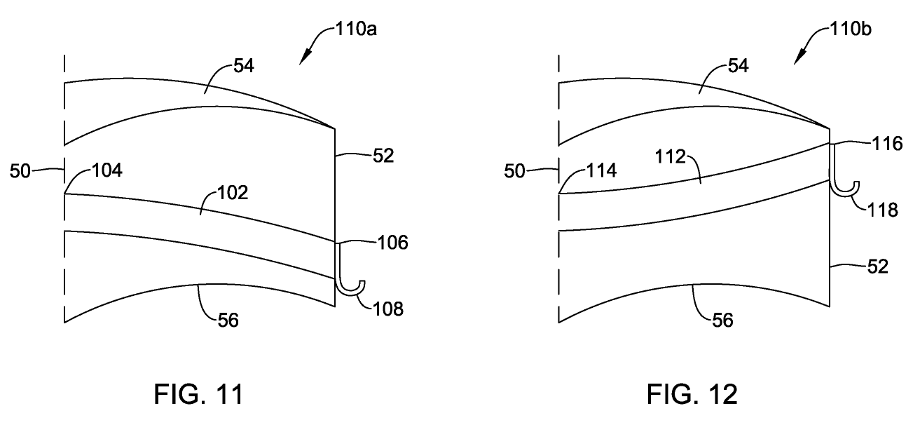

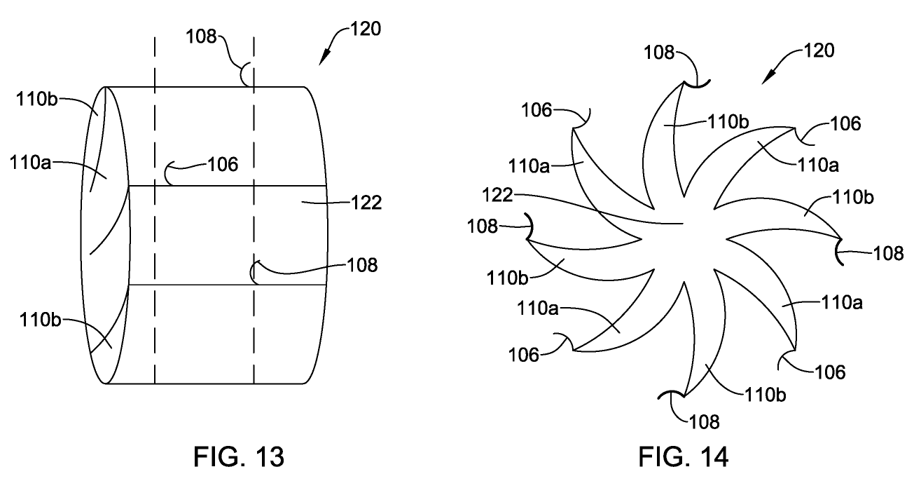

FIG. 11 shows a wing 110a having a structural component 102 extending from a first end 104 proximate the central region 50 to a free end 106 proximate the periphery 52. The structural component 102 includes an anchor 108 secured to the free end 106. FIG. 12 shows a wing 110b having a structural component 112 extending from a first end 114 proximate the central region 50 to a free end 116 proximate the periphery 52. The structural component 112 includes an anchor 118 secured to the free end 116. In some cases, the structural component 102 and/or the structural component 112 may be concave or convex in shape. The structural component 102 and the structural component 112 may both curve in a same direction, or in an opposite direction. In some cases, a medical device including the structural component 102 and the structural component 112 may have anchors at a plurality of different axial “latitudes”. FIGS. 13 and 14 are schematic views of an illustrative medical device 120 that includes a foam body 122 that is formed of alternating wings 110a and 110b. FIG. 13 is a side view while FIG. 14 is a top view. In some cases, the medical device 120 may include a variety of different structural components, including any of the structural components 28, 30, 58, 64, 72, 84, 102, and 104. The structural components 28, 30, 58, 64, 72, 84, 102, and 104 may in combination be considered as forming a structural member, regardless of whether the individual structural components 28, 30, 58, 64, 72, 84, 102, and 104 are secured together or are merely disposed within the foam.

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha,omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Patent Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted for occluding a patient’s left atrial appendage (LAA), the LAAC device comprising:
a shape memory polymer foam body adapted to expand from a crimped configuration for delivery to an expanded configuration after delivery; and
a structural member extending radially within the shape memory polymer foam body that is adapted to assist in radial expansion of the shape memory polymer foam body.

2. The LAAC device of claim 1, wherein the shape memory polymer foam body comprises a shaped memory polymer foam body.

3. The LAAC device of claim 1, wherein the shape memory polymer foam body comprises a plurality of wings formed within the shape memory polymer foam body.

4. The LAAC device of claim 3, wherein the structural member comprises a plurality of structural components extending through at least some of the plurality of wings.

5. The LAAC device of claim 3, wherein the structural member extends radially within one or more of the plurality of wings.

6. The LAAC device of claim 5, wherein the structural member extends radially from a central hub.

7. The LAAC device of claim 3, wherein the structural member also extends axially within one or more of the plurality of wings.

8. The LAAC device of claim 3, wherein the structural member extends internally within one or more of the plurality of wings.

9. The LAAC device of claim 3, wherein the structural member extends externally to one or more of the plurality of wings.

10. The LAAC device of claim 1, wherein the structural member comprises a shape memory material.

11. The LAAC device of claim 9, wherein the structural member comprises a shape memory metal.

12. The LAAC device of claim 9, wherein the structural member comprises a shape memory polymer.

13. The LAAC device of claim 3, wherein the structural member extends radially through one or more of the plurality of wings to a periphery thereof.

14. The LAAC device of claim 13, wherein the structural member further comprises an anchor extending radially from the periphery of the one or more of the plurality of wings.

15. A medical device, comprising:
a shape memory polymer foam body adapted to expand from a crimped configuration for delivery to an expanded configuration after delivery; and
a structural member extending radially within the shape memory polymer foam body that is adapted to assist in radial expansion of the shape memory polymer foam body.

16. The medical device of claim 15, wherein the structural member comprises a shape memory metal.

17. The medical device of claim 15, wherein the structural member comprises a shape memory polymer.

18. The medical device of claim 15, further comprising an anchor secured to a free end of the structural member.

19. The medical device of claim 15, wherein the shape memory polymer foam body is adapted to occlude a left atrial appendage (LAA).

20. A left atrial appendage closure (LAAC) device adapted for occluding a patient’s left atrial appendage (LAA), the LAAC device comprising:
a shaped shape memory polymer foam body adapted to expand from a crimped configuration for delivery to an expanded configuration after delivery, the shaped shape memory polymer foam body including a radially extending wing;
a structural member extending radially within the radially extending wing; and
an anchor extending from the structural member.
